(19) United States
(12) Patent Application Publication
(10) Pub. No.: US 2013/0045266 A1
(43) Pub. Date: Feb. 21, 2013

(54) METHOD FOR PREPARING POLYMERIC BIOMATERIALS HAVING IMMOBILIZED DRUG DELIVERY SYSTEM COMPRISING BIOACTIVE MOLECULES LOADED PARTICLE CARRIER

(71) Applicants: Seok Hwa CHOI, Cheongju-si (KR); Jun Sik SON, Gwangju (KR); Kyu-Bok LEE, Daegu (KR); Seong Soo KANG, Gwangju (KR)

(72) Inventors: Seok Hwa CHOI, Cheongju-si (KR); Jun Sik SON, Gwangju (KR); Kyu-Bok LEE, Daegu (KR); Seong Soo KANG, Gwangju (KR)

(73) Assignees: CHUNGBUK NATIONAL UNIVERSITY INDUSTRY ACADEMIC COOPERATION FOUNDATION, Cheongju (KR); INDUSTRY FOUNDATION OF CHONNAM NATIONAL UNIVERSITY, Gwangju (KR); KYUNGPOOK NATIONAL UNIVERSITY INDUSTRY-ACADEMIC COOPERATION FOUNDATION, Daegu (KR)

(21) Appl. No.: 13/586,740

(22) Filed: Aug. 15, 2012

(30) Foreign Application Priority Data
Aug. 16, 2011 (KR) 10-2011-0081130

(51) Int. Cl. B05D 7/00; A61P 17/02; A61P 19/08; A61P 29/00; A61P 31/04; B05D 3/02; A61K 9/70; A61P 3/00; B82Y 5/00

(52) U.S. Cl. 424/422; 427/2.24; 427/2.31; 427/2.26; 977/773; 977/788; 977/906

(57) ABSTRACT

A new polymeric material for biological tissue regeneration or treatment with a drug delivery system which contains therapeutic agents and/or bioactive molecules to reduce infection and inflammatory reaction at a wound site and maximize tissue regeneration and wound healing is provided. The new bioactive molecule-loaded polymeric material is prepared by (1) preparing micrometer or nanometer sized bioactive molecule-loaded particles; (2) modifying the surface of the prepared particles and immobilizing the particles on the surface of the polymeric material; and (3) physically treating the surface of the polymeric material to improve binding strength of the particles immobilized thereon.

### CROSS-REFERENCE TO RELATED APPLICATIONS

Pursuant to 35 U.S.C. §119(a), this application claims the benefit of earlier filing date and right of priority to Korean Patent Application No. 10-2011-0081130, filed on Aug. 16, 2011, the contents of which are hereby incorporated by reference herein in its entirety.

### BACKGROUND OF THE INVENTION

1. Field of the Invention

The present invention relates to a new polymeric material for biological tissue regeneration and treatment with a particle drug delivery system and a method for preparing the same. More particularly, the present invention relates to a polymeric material prepared by immobilizing particles, prepared by loading bioactive molecules into polymeric particles having a size of several tens of nanometers to several hundreds of micrometers, or particles, prepared using a polyelectrolyte or ionic complex of bioactive molecules having a negative or positive charge, on the surface of the polymeric material. The prepared polymeric material may be easily and rapidly applied regardless of the type of raw material for the polymeric material and the shape thereof. Moreover, a variety of bioactive molecules may be effectively incorporated into the surface of the polymeric material without affecting the intrinsic properties of the polymeric material (such as mechanical strength, in-vivo breaking strength, knot strength, degradation rate, surface modification, porosity, etc.). Furthermore, the present invention relates to a method for preparing a new bioactive molecule-loaded polymeric material, which is slowly released from particles incorporated into the surface of the polymeric material, and a polymeric material with a drug delivery system prepared by the method, which can solve the problems of conventional polymeric materials for human application, such as low biocompatibility, low therapeutic effect, inflammatory response, and foreign body reaction, and significantly improve wound healing and tissue regeneration.

2. Description of the Related Art

Biomaterials are materials that are in direct contact with biological tissues and can be produced and distributed after proper verification of biosafety and clinical trials by tosicological, pathological, and physiological examinations. Such biomaterials are classified into those having physical functions, such as mechanical properties (e.g., strength, elasticity, etc.), gas exchange (in an artificial lung), and adhesive properties, and those having physiological (chemical) functions such as biosafety (e.g., elution of impurities, cytotoxicity, antigenicity, oncogenicity, etc.) and biocompatibility (e.g., bulk biocompatibility, thrombosis, etc.). For the last several decades, the physical functions of the polymeric materials for human application have been significantly improved due to the development of various materials, but the physiological functions for tissue regeneration and wound healing have been less developed. Specifically, in recent times, as tissue engineering using stem cells existing in a human body (human adipose tissue-derived stem cells, human umbilical cord-derived stem cells, human amnion-derived stem cells, bone marrow stem cells, mesenchymal stem cells, etc.) has been studied, the relationship between the polymeric material and the stem cells has been studied for more effective tissue regeneration and wound healing by inducing proliferation and differentiation of the stem cells in the polymeric material. As a result, the physiological functions of the polymeric material become more important. A typical method for improving the physiological functions of the polymeric material is to incorporate transforming growth factors of the stem cells or several bioactive molecules, which are effective in tissue regeneration and wound healing, into the surface of the polymer. The key point of this method is to incorporate bioactive molecules on the polymeric material suitable for human application in the most appropriate method such that the incorporated drugs are released in an amount that can achieve a stable treatment effect.

Most of the currently studied techniques relate to a method of directly connecting bioactive molecules with a polymer having a functional group by chemical bonding through a complex chemical treatment [Jeon et al. Biomaterials, 28, 2763 (2007), Lin et al, Biomaterials, 29, 1189 (2008)], or a method of mixing bioactive molecules with a polymer [Chiu et al, J Biomed Mater Res, 83A, 1117 (2007), Fu et al, Biotechnol Bioeng, 99, 996 (2008), Yoon et al, Biomaterials, 24, 2323 (2003)]. However, these methods have disadvantages such as the cytotoxicity of the polymer having the functional group, the probability of remaining chemicals, the complex processing, and the probability of a modified surface of the polymeric material. Particularly, it is difficult to control the releasing rate and amount of the bioactive molecules of the thus prepared polymeric material, and the bioactivity of the bioactive molecules may be significantly reduced during chemical treatments.

Thus, the inventors of the present invention have conducted intensive studies to prepare a functional polymeric material for tissue regeneration and wound healing with a drug delivery system by a new method and, as a result, found that when biodegradable polymeric particles into which bioactive molecules were loaded or particles consisting of bioactive molecules were immobilized on the surface of a polymeric material in a physical manner or inserted into pores of the polymeric material and immobilized, various types of bioactive molecules were easily incorporated into the surface of the polymeric material while maintaining the intrinsic properties of the polymeric material suitable for humans, the particles immobilized into the surface of the polymer were biodegraded, the bioactive molecules were continuously released, and thereby it was possible to improve cytotropism, reduce inflammation, and more effectively induce the wound healing and tissue regeneration, thus completing the present invention.

### SUMMARY OF THE INVENTION

The present invention is directed to a new polymeric material with a drug delivery system and a method for preparing the same. In order to improve low histocompatibility and wound healing effect of conventional polymeric materials, bioactive molecule-loaded particles are incorporated into the surface of the polymeric material to prepare a bioactive molecule-loaded functional polymeric material, which maximizes the therapeutic effect without any change in intrinsic properties of the polymeric material. The polymeric material is prepared by preparing particles by loading bioactive molecules into polymeric particles having a size of several tens of nanometers to several hundreds of micrometers or preparing particles using a polyelectrolyte or ionic complex of bioactive molecules having a negative or positive charge and immobilizing the particles on the surface of the polymeric material in a physical manner. The thus prepared polymeric material may be easily and rapidly applied regardless of the type of raw material for the polymeric material and the shape thereof. Moreover, a variety of bioactive molecules may be simply and effectively incorporated into the surface of the polymeric material without affecting the intrinsic properties of the polymeric material. Furthermore, the present invention relates to a method for preparing a new polymeric material with a drug delivery system, in which bioactive molecules are continuously released from particles incorporated into the surface of the polymeric material, not affecting the intrinsic properties of the polymeric material even after the incorporation of bioactive molecules, to improve cytotropism, reduce inflammation, and more effectively induce the wound healing and tissue regeneration, thus supporting active tissue regeneration.

In an aspect, the present invention provides a method for preparing a functional polymeric material with a drug delivery system, the method including: (1) preparing micrometer or nanometer sized bioactive molecule-loaded particles; (2) modifying the surface of the prepared particles and immobilizing the particles on the surface of the polymeric material; and (3) physically treating the surface of the polymeric material to improve binding strength of the particles immobilized thereon.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIGS. 1A and 1B are scanning electron microscope (SEM) images of the surfaces of biodegradable PDO sutures on which bioactive molecule-loaded PLGA particles prepared according to a method of the present invention are immobilized, respectively;

FIG. 2A is an SEM image showing that bioactive molecule-loaded PLGA nanoparticles prepared according to a method of the present invention are immobilized on the surface of a non-degradable silk suture (braised) and a gap between fibers, and FIG. 2B is an high-magnification SEM image showing improved binding strength between nanoparticles and the surface of a suture after post-treatment;

FIG. 3A shows a collagen film for GBR on which bioactive nanoparticles are immobilized by ion complex prepared according to a method of the present invention, and FIG. 3B is an SEM image showing that multiple bioactive molecule-loaded biodegradable polymer particles are immobilized on a collagen film;

FIG. 4 is an SEM image showing PGA polymer films for GTR on which bioactive nanoparticles and bioactive molecule-loaded biodegradable polymeric particles prepared by ion complex according to a method of the present invention are immobilized;

FIG. 5A shows a biodegradable PLGA polymeric scaffold on which multiple bioactive molecule-loaded biodegradable polymeric particles prepared according to a method of the present invention are immobilized, and FIG. 5B is an SEM image showing apatite coated on the surface a scaffold on which particles are immobilized scaffold by a biomimetic process; and

FIG. 6 is an SEM image showing that bioactive nanoparticles prepared by ion complex according to a method of the present invention are immobilized between biodegradable polymeric PCL nanofibers membrane.

### DETAILED DESCRIPTION OF THE INVENTION

Hereinafter, exemplary embodiments of the present invention will be described in detail below with reference to the accompanying drawings such that those skilled in the art to which the present invention pertains can easily practice the present invention.

The present invention relates to a new polymeric material with a drug delivery system prepared by effectively incorporating bioactive molecules into a polymeric material to improve wound healing and tissue regeneration at an implantation site.

Hereinafter, the present invention will be described in detail.

A method for preparing a polymeric material with a drug delivery system according to the present invention includes:

(1) preparing micrometer or nanometer sized bioactive molecule-loaded particles;

(2) modifying the surface of the prepared particles and immobilizing the particles on the surface of the polymeric material; and

(3) physically treating the surface of the polymeric material to improve binding strength of the particles immobilized thereon.

Step (1) is to prepare micrometer or nanometer sized bioactive molecule-loaded particles and includes: (A) preparing particles using a biodegradable polymer; and (B) preparing particles by ion complex of bioactive molecules having a charge. First, in Step 1-A, the bioactive molecule-loaded biodegradable polymeric particles may have a diameter ranging from several nanometers to several micrometers and may be prepared by a water/oil or water/oil/water emulsion method, a spray method, or a phase separation method. The particles may be prepared in the form of a circle or powder, and preferably, biodegradable polymeric particles, which are prepared by the emulsion method and non-porous or have a porosity of 5 to 98% and a diameter of 10 nm to 1 mm, are used. In Step 1-B, the particles prepared by ion complex of bioactive molecules having a charge may have a diameter ranging from several nanometers and several micrometers and may use any bioactive molecule having a charge. That is, any bioactive molecules having a positive or negative charge which can induce a polyelectrolyte complex by different charges of two types of bioactive molecules after being stirred or left in an appropriate solution and a potential of hydrogen (pH) may be used. The particles may be prepared in the form of a circle or powder, and preferably, bioactive molecule-loaded particles, which are non-porous or have a porosity of 5 to 98% and a diameter of 10 nm to 500 μm, may be used.

Raw materials for the polymeric material used in the present invention may include, but are not limited to, at least one selected from the group consisting of silk, cotton, linen, collagen, chitin/chitosan, polydioxanone (PDO), poly(glycolic acid) (PGA), polylactic acid (PLA), poly(ε-caprolactone) (PCL), lactide-co-glycolic acid (PLGA), glycolide-co-trimethylene carbonate (GA-TMC), glycolide-co-ε-caprolactone (GA-CL), polyglyconate (PGC), polyglactin (PG), polyamino acid, polyanhydride, polyorthoester, a polyester group, a polyether group, a polyethylene group, a polypropylene group, a polybutester group, a polytetrafluoroethylene group, a polyamide group, a polyimide group, polyvinylidene fluoride and copolymers thereof, stainless steel, titan, nitinol, silver, gold, and mixtures or complexes thereof.

The polymeric material for human application using the above-mentioned raw materials may be in the form of, for example, a block, a film, a filament, a fiber, non-woven/woven, a membrane, a mesh, a knit, a granule, a particle, a plate, a bolt/screw, a pin, or a complex thereof.

When these polymeric materials are applied to human, any polymeric materials for tissue regeneration or treatment which are in direct contact with a living human tissue may be used. The polymeric materials may include: but are not limited to, surgical suture and ligature strands; scaffolds and patches for soft and hard tissue regeneration, guided bone regeneration (GBR), and guided tissue regeneration (GTR); surgical meshes; and gauze for wound dressing, artificial organs and vessels, plates, bolts, screws, or pins.

Raw materials for the particles used in the present invention may include any materials capable of being used for human application and include: for example, polydioxanone (PDO), poly(glycolic acid) (PGA), polylactic acid (PLA), poly(ε-caprolactone) (PCL), lactide-co-glycolic acid (PLGA), glycolide-co-trimethylene carbonate (GA-TMC), glycolide-co-ε-caprolactone (GA-CL), polyglyconate (PGC), polyglactin (PG), polyamino acid, polyanhydride, polyorthoester, and copolymers thereof; collagen, gelatin, chitin/chitosan, alginate, albumin, hyaluronic acid, heparin, fibrinogen, cellulose, dextran, pectin, polylysine, polyethyleneimine, dexamethasone, chondroitin sulfate, lysozyme, DNA, RNA, protein derivatives, and copolymers thereof; growth factors, growth hormones, peptide drugs, protein drugs, anti-inflammatory and analgesics drugs, anti-cancer drugs, anti-viral drugs, sex hormones, antibiotics, antimicrobials, and compounds thereof; and metals such as gold, silver and zinc. Among them, polylactic acid (PLA), polydioxanone (PDO), poly(glycolic acid) (PGA), lactide-co-glycolic acid (PLGA) or copolymers or mixtures thereof; or natural polymers, protein derivatives or mixtures thereof, which are approved by the FDA of the U.S.A. as biodegradable polymers capable of being used for human may be used, but the present invention is not limited thereto.

The bioactive molecules capable of being used in the present invention may include growth factors, growth hormones, peptide drugs, protein drugs, anti-inflammatory and analgesics drugs, anti-cancer drugs, anti-viral drugs, sex hormones, antibiotics, antimicrobials, and compounds thereof. Examples of the bioactive molecules may include growth factors such as a transforming growth factor, (TGF), a fibroblast growth factor (FGF), a bone morphogenic protein (BMP), a vascular endothelial growth factor (VEGF), an epidermal growth factor (EGF), an insulin-like growth factor (IGF), a platelet-derived growth factor (PDGF), a nerve growth factor (NGF), a hepatocyte growth factor (HGF), a placental growth factor (PIGF), and a granulocyte colony stimulating factor (G-CSF); peptide and protein drugs such as heparin, porcine growth hormone (pGH), human growth hormone (hGH), erythropoietin, (EPO), a granulocyte colony stimulating factor (gCSF), interferon (INF), follicle stimulating hormone (FSH), luteinizing hormone (LH), goserelin acetate, leuprorelin acetate, triptorelin acetate, and luteinizing hormone-releasing hormone agonist (LH-RH agonist); anti-inflammatory and analgesics drugs such as dexamethasone, indomethacin, ibuprofen, ketoprofen, piroxicam, flurbiprofen, and diclofenac; anti-cancer drugs such as paclitaxel, doxorubicin, camptothecin, 5-fluorouracin, cytosine arabinose, and methotrexate; anti-viral drugs such as acyclovir, Robavin, and Tamiflu; sex hormones such as testosterone, estrogen, progesterone, and estradiol; antibiotics such as tetracycline, minocycline, doxycycline, ofloxacin, levofloxacin, ciprofloxacin, clarthromycin, erythromycin, cefaclor, cefotaxim, imipenem, enicillin, gentamicin, streptomycin, and vancomycin; anti-fungal drugs such as ketoconazole, itraconazole, fluconazole, amphotericin-B, mystatin, and griseofulvin; and compounds such as β-glycerophosphate, ascorbate, hydrocortisone, and 5-azacytidine. These bioactive molecules may be loaded in polymeric particles or particles may be prepared by ion complex of bioactive molecules. Preferably, the bioactive molecules may be included in the particles in the range from 10⁻⁷% to 100% with respect to the total weight of the particles.

Step (2) is to modify the surface of the prepared particles and immobilize the particles on the surface of the polymeric material and includes: (C) modifying the surface charge of the prepared particles to be opposite to the polymeric material and physically immobilizing the particles on the surface of the polymeric material by electrostatic interaction; (D) partially dissolving the surface of the particles by dispersing the prepared particles in an appropriate solvent to be immobilized on the surface of the polymeric material; and (E) inserting the particles into pores or gaps of the polymeric material to be immobilized.

In more detail, in Step 2-C, when the surface of the prepared particles has a negative charge and the surface of the polymeric material to be used has a positive charge, it is possible to disperse the particles in an appropriate solution or aqueous solution and immerse the polymeric material in the resulting solution to induce electrostatic interaction such that the particles are immobilized on the surface of the polymeric material without any surface modification. Alternatively, when the surface of the prepared particles has a negative charge and the surface of the polymeric material to be used also has a negative charge, it is possible to coat the negatively-charged particles with an appropriate material having a positive charge and allow the resulting particles to be immobilized on the surface of the polymeric material by the electrostatic interaction in an appropriate solution or aqueous solution.

In Step 2-D, when the polymeric particle is composed of a biodegradable polymer, it is possible to disperse the particles in an appropriate solvent capable of partially dissolving the biodegradable polymer such that the surface of the particles is partially dissolved, thereby immobilizing the biodegradable polymeric particles on the surface of the polymeric material.

In Step 2-E, when the polymeric material has pores or gaps, it is possible to insert the particles into the pores or gaps of the polymeric material without using methods C and D or direct surface modification of the particles, and it is possible to immobilize the particles by an appropriate method, for example, using a solvent or heat.

Materials for coating the particles used in Step 2 may include any ionic polymer having either a negative or positive charge and may include, but are not limited to, at least one selected from the group consisting of polydioxanone (PDO), poly(glycolic acid) (PGA), polylactic acid (PLA), poly(ε-caprolactone) (PCL), lactide-co-glycolic acid (PLGA), glycolide-co-trimethylene carbonate (GA-TMC), glycolide-co-ε-caprolactone (GA-CL), polyglyconate (PGC), polyglactin (PG) and copolymers thereof; collagen, heparin, albumin, hyaluronic acid, dextran, vancomycin, chitosan, dexamethasone, chondroitin sulfate, lysozyme, polylysine, polyethyleneimine (PEI), sodium tripolyphosphate (TPP), polystyrene sulfonate (PSS), polyallylamine (PAAm), polyvinylamine (PVAm), poly(diallyldimethylammonium chloride) (PDADMAC), poly(methylamino) ethyl methacrylate (PDAMAEMA), N-hydroxysuccinimide (NHS), and N-3-dimethylaminopropyl-N′-ethyl-carbodiimide hydrochloride (EDC), and combinations thereof.

The solution and aqueous solution which can be used in Step 2 may be any one which does not significantly affect the properties of the surface charge characteristic of the polymeric material or the particles in the solution without limitation. Preferably, water, ethanol, acetone, methanol, heptane, pentane or a mixed solution thereof may be used in a pH of 2 to 9.

The solvent capable of partially dissolving the polymeric particles in Step 2, while it depends on the raw material for the polymer, may include water, hydrochloric acid, acetic acid, methylene chloride, ethanol, acetone, methanol, dichloromethane, chloroform, toluene, acetonitrile, 1,4-dioxane, tetrahydrofuran, hexafluoroisopropanol or a mixed solvent thereof. Preferably, ethanol, acetone, methanol or water is used in a weight ratio of 0 to 100 wt %.

In Step (2), plasma treatment may be performed for surface hydrophilicity of the particles prepared using the biodegradable polymer or the polymeric material and activation of the surface charge thereof. The modification of the surface hydrophilicity and the activation of the surface charge by the plasma treatment may improve the electrostatic interaction between the particles and the polymeric material, but the plasma treatment is not necessarily performed. A gas which can be used in the plasma treatment may be oxygen, argon, hydrogen peroxide, or ammonia, and preferably oxygen and argon are used.

Step (3) is to physically treat the surface of the polymeric material to improve binding strength of the particles immobilized thereon. This step may depend on the polymer used as a raw material for the particles, and this step is not necessarily performed with respect to all polymeric materials on which particles are immobilized. However, this step may be performed to improve the binding strength between the particles and the surface of the polymeric material or increase the bioactivity of the surface of the polymeric material. Step (3) to improve the binding strength between the particles and the surface of the polymeric material includes: (F) treating the particles or the polymeric material with a solvent capable of partially dissolving the particles or the surface of the polymeric material; (G) partially melting the particles or the surface of the polymeric material by heat; and (H) forming apatite on the surface of the polymeric material by a biomimetic process.

In more detail, Step 3-F is to improve the binding strength between the particles and the surface of the polymeric material by immersing the particle-immobilized polymeric material, prepared in step (2), in the solvent capable of partially dissolving the particles or the polymeric material suggested in step (2) for a predetermined time. Here, the immersion time in the solvent may depend on the used polymer, and preferably the polymeric material is immersed to the extent that the properties of the bioactive molecules loaded into the particles or those of the polymeric material are not degraded. More preferably, when the particles or polymeric material is composed of a biodegradable polymer, the polymeric material is immersed in an aqueous solution prepared by mixing ethanol, methanol, acetone, and acetic acid with water in a weight ratio of 0 to 100 wt % for 1 minute to 2 hours and dried at room temperature or freeze-dried. Step 3-F may be performed when the particles or the polymeric material is a thermoplastic polymer or when the bioactive molecules loaded in the particles are sensitive to an organic solvent. In Step 3-F, both dry and wet heat may be used, and the range and time of heating depend on the used polymer. Preferably, Step 3-F may be performed above the glass transition temperature and below the melting temperature, and preferably, the particle-immobilized polymeric material, prepared in step (2), is treated in the temperature range of 30 to 300° C. for ten seconds to 1 hour.

Step 3-H is to coat the surface of the polymeric material on which the bioactive molecule-loaded particles are immobilized, prepared in step (2), with apatite by a biomimetic process. While this step is not necessarily performed, it may be performed to increase the bioactivity of the surface of the polymeric material for soft tissue regeneration. The particle-immobilized polymeric material is immersed in a simulated body fluid (SBF) solution at 37±0.5° C. and in a pH of 6.4 to 7.4 for 1 hour to 30 days. Here, 1×SBF solution is prepared by adding 8.035 g NaCl, 0.355 g NaHCO, 0.225 g KCl, 0.231 g K₂HPO₄.3H₂O, 0.311 g MgCl₂.6H₂O, 0.292 g CaCl₂, 0.072 g Na₂SO₄, 6.118 g [(HOCH₂)₃(CNH₂)] and 39 ml of 1.0 M HCl to 1 L of distilled water, and preferably it may be used at a concentration of 1 to 5 times.

In order to rapidly form the apatite on the surface of the polymeric material, an alternate dipping process may be performed in which the polymeric material is immersed once or five times in a 1 to 90% ethanol aqueous solution, in which calcium chloride (CaCl₂) and dipotassium phosphate (K2HPO₄) are dissolved, to a precursor of apatite on the surface thereof. Preferably, after each 0.1 M of calcium chloride and dipotassium phosphate is dissolved in a 50% ethanol aqueous solution, the polymeric material is immersed in 0.1 M calcium chloride-ethanol aqueous solution for 10 seconds, immersed in a pure 50% ethanol aqueous solution for 1 second, dried at room temperature for 3 minutes, immersed again in 0.1M dipotassium phosphate-ethanol aqueous solution for 10 seconds, immersed in a pure 50% ethanol aqueous solution for 1 second, and then dried at room temperature for 3 minutes, which are repeated three times.

The thus prepared new bioactive molecule-loaded polymeric material can be applied in the present invention regardless of the type of raw material for the polymeric material and the shape thereof, and a variety of bioactive molecules can be simply and effectively incorporated into the surface of the polymeric material. Moreover, it is possible to control the content of bioactive molecules, released from the particles on the surface of the polymeric material, and the releasing rate and time of the bioactive molecules, and thus it is possible to prepare a functional polymeric material with a drug delivery system, which significantly improves the cytotropism, inflammatory reaction and wound healing at an implantation site. The functional polymeric material can be effectively used in an implantable biomedical device capable of supporting active tissue regeneration.

Hereinafter, the present invention will be described in further detail with reference to the following exemplary embodiments. It should be understood that the exemplary embodiments are merely illustrative and not restrictive of the present invention.

### Exemplary Embodiment 1

To begin with, as a polymeric material for the test, a commercially available polydioxanone (PDO) monofilament suture was prepared. Particles using a biodegradable polymer were prepared as follows. 1 g of PLGA (75:25) was completely dissolved in 9 ml of dichloromethane, and 40 mg of dexamethasone as a bioactive molecule was dissolved in 1 ml of ethanol. The resulting solution was put into the PLGA solution and stirred for 20 minutes, thereby preparing a PLGA polymer solution containing dexamethasone. Subsequently, the thus prepared dexamethasone/PLGA polymer solution was mixed with 100 ml of a 0.2% polyvinyl alcohol (PVA) aqueous solution and was stirred with a homogenizer at 10,000 rpm for 3 minutes. Then, the resulting solution was poured into 300 ml of a 0.5% PVA aqueous solution and was stirred at 600 rpm for 5 hours. The resulting product was washed with distilled water three times and was freeze-dried, thereby preparing dexamethasone/PLGA particles.

Hydrophilic surface modification of the dexamethasone/PLGA particles was performed using oxygen plasma at 18 Watts for 3 minutes. To coat the hydrophilic-modified surface of the PLGA particles with a positively-charged material, the PLGA particles were dispersed in a 0.05 wt % PEI aqueous solution and stirred for 12 hours. Subsequently, the PLGA particles coated with the PEI were collected using a centrifugal separator and washed three times to remove unreacted PEI, and the resulting PLGA particles were freeze-dried.

To immobilize the PEI-coated dexamethasone/PLGA particles on the surface of the PDO suture having a negatively-charged surface, a certain amount of PLGA particles coated with positively-charged polymers, PEI, was dispersed in deionized water, and the PDO suture was put therein. The resulting solution was slowly shaken for 4 hours and then washed with distilled water three times to remove excess PLGA particles which were not immobilized on the surface of the PDO suture. Subsequently, in order to improve the binding strength between the PLGA particles and the surface of the PDO suture, after completely drying the PDO suture at room temperature, the PDO suture was immersed in an 80% ethanol aqueous solution for 3 minutes and was dried at room temperature to give a dexamethasone-loaded PLGA particle-immobilized PDO suture with a drug delivery system.

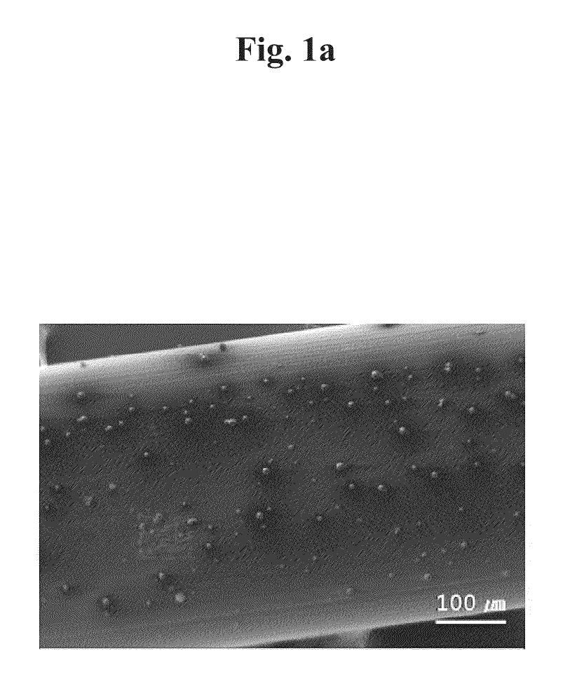

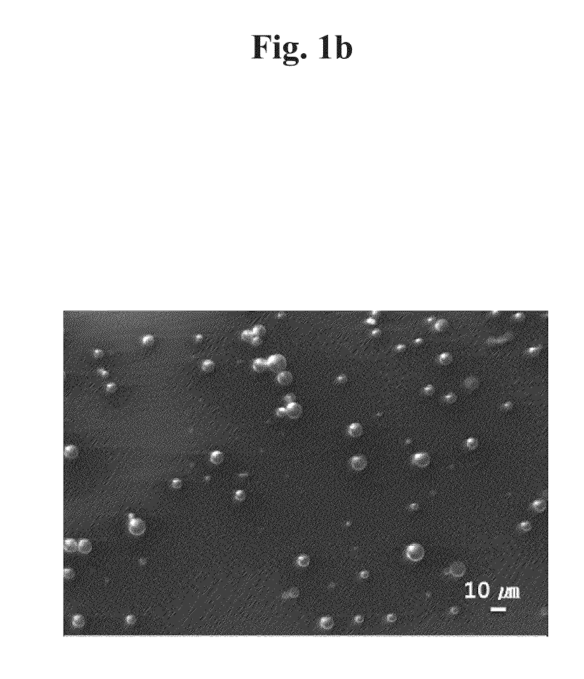

The morphology of the dexamethansone-loaded PLGA particle-immobilized PDO suture was observed on a scanning electron microscope (SEM), and the results are shown in FIGS. 1A and 1B. It was observed that the average size of the dexamethasone-loaded PLGA particles was 6 μm and the PEI-coated PLGA particles were uniformly dispersed on the surface of the PDO suture.

### Exemplary Embodiment 2

As a polymeric material for the test, a commercially available silk suture (#3, braided) was prepared. PLGA nanoparticles were prepared as follows. 200 mg of PLGA (75:25) was completely dissolved in 10 ml of dichloromethane, and 40 mg of dexamethasone as a bioactive molecule was dissolved in 1 ml of ethanol. The resulting solution was put into the PLGA solution and stirred for 20 minutes, thereby preparing a dexamethasone-loaded PLGA polymer solution. Subsequently, the thus prepared dexamethasone/PLGA polymer solution was mixed with 25 ml of a 0.5% PVA aqueous solution and was stirred with a homogenizer at 10,000 rpm and for 3 minutes. Then, the resulting solution was poured into 65 ml of a 0.5 PVA aqueous solution and stirred at 700 rpm for 5 hours. In the same manner as Exemplary Embodiment 1, the dexamethasone-loaded PLGA nanoparticles were immobilized on the surface of the silk suture. Then, heat treatment was performed at 150° C. for 10 seconds to improve binding strength between them.

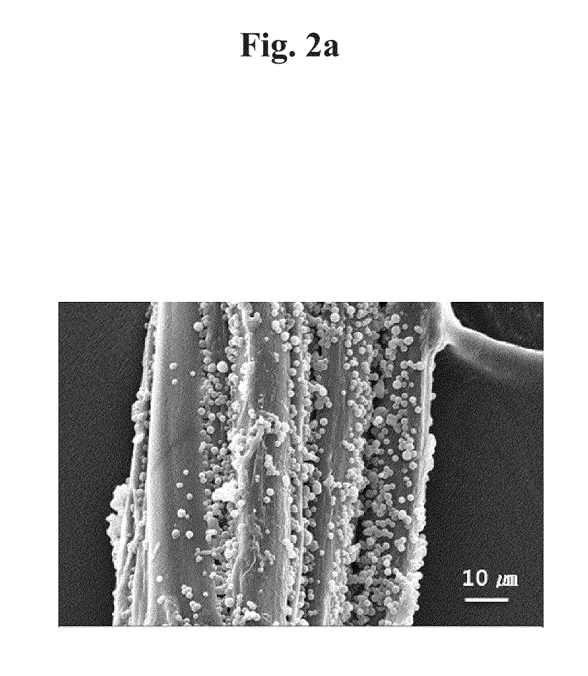

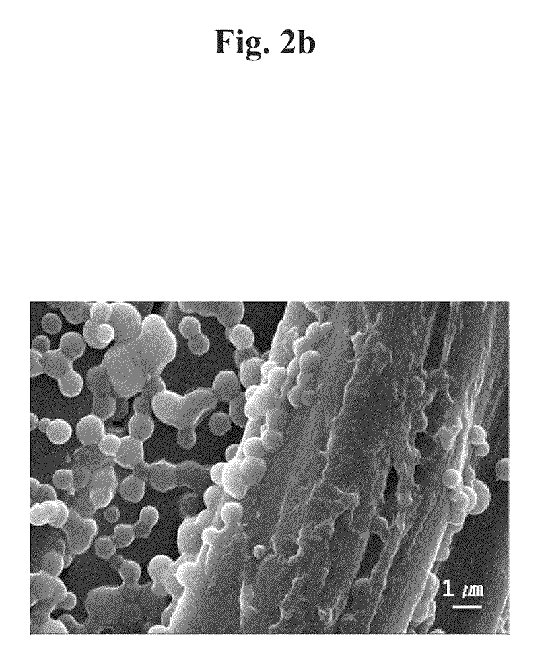

The morphology of the silk suture on which the dexamethasone-loaded PLGA nanoparticles were immobilized was observed on an SEM, and the results are shown in FIGS. 2A and 2B. It was confirmed that the PLGA nanoparticles uniformly were dispersed on the surface of the silk suture and in a gap between fibers of the silk suture, and it was indirectly confirmed from the high-magnification SEM images that the binding strength between them was improved by heat treatment.

### Exemplary Embodiment 3

As a polymeric material for the test, a collagen film for GBR was prepared. To prepare polyelectrolyte nanoparticles, 0.1% vancomycin, an antibiotic, as a positively-charged material and a 0.1% dexamethasone disodium phosphate aqueous solution effective in inhibiting inflammation and tissue regeneration as a negatively-charged material were prepared. The dexamethasone aqueous solution was added dropwise to the vancomycin aqueous solution and was shaken for 2 hours, thereby preparing vancomycin/dexamethasone nanoparticles by ion complex. To remove unreacted vancomycin and dexamethasone, the vancomycin/dexamethasone nanoparticles were filtered through a permeable membrane and were centrifuged to harvest. Then, a predetermined amount of the nanoparticles was dispersed in deionized water, a collagen film was added thereto, and the resulting solution was slowly shaken for 5 hours to give a collagen film for GBR with a drug delivery system on which vancomycin/dexamethasone nanoparticles were immobilized on the surface thereof.

After each of dexamethasone and vancomycin-loaded PLGA particles was prepared in the same manner as Exemplary Embodiment 1, the collagen film was put in distilled water in which the two types of particles were mixed together in a ratio of 50:50 wt % and dispersed and then slowly shaken for 4 hours to give a collagen film on which PLGA/dexamethasone and PLGA/vancomycin particles were immobilized.

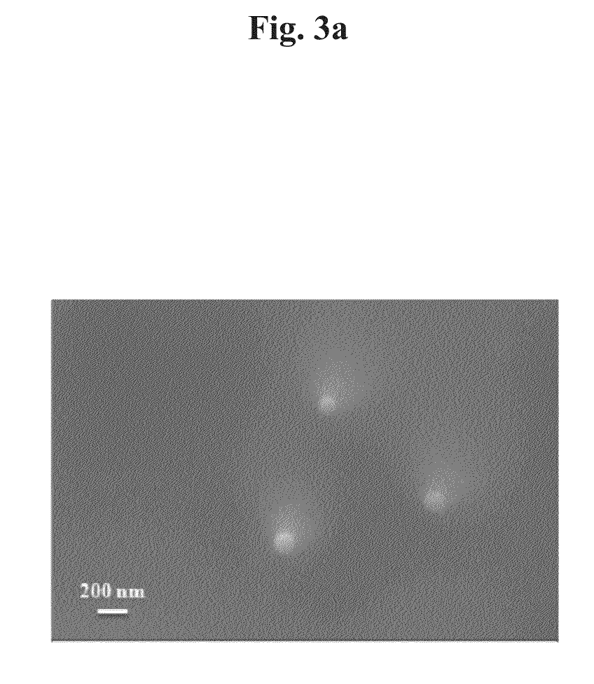

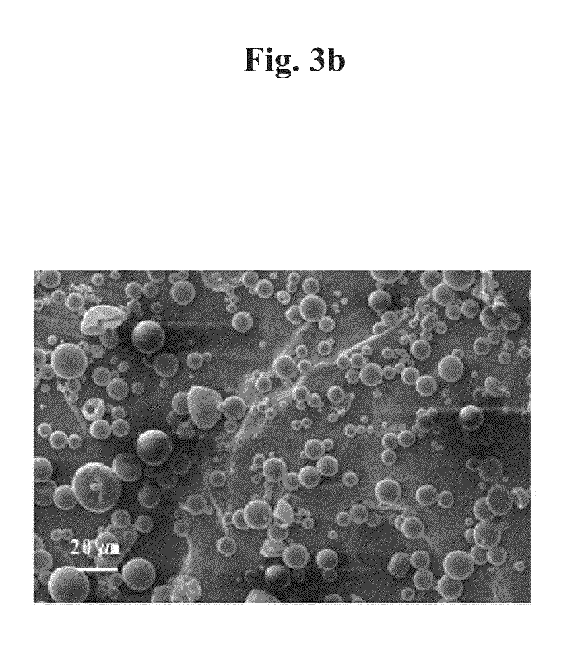

It could be seen from ζ-potential analyses that the average size of the nanoparticles was 200 nm and the surface of the nanoparticles was composed of positively-charged vancomycin molecules. It could be confirmed that the dexamethasone and vancomycin nanoparticles as shown in FIG. 3A and the PLGA/dexamethasone and PLGA/vancomycin nanoparticles as shown in FIG. 3B were immobilized and well dispersed on the collagen film having a negatively-charged surface.

### Exemplary Embodiment 4

As a polymeric material for the test, a PGA film for GRT was prepared. PGD-peptide-loaded PLGA nanoparticles as a bioactive molecule were prepared as follows. First, 200 mg of PLGA was completely dissolved in 10 ml of chloroform, and 2 ml of an aqueous solution, in which 20 mg of RGD was dissolved, was mixed with the PLGA polymeric solution and stirred with a homogenizer at 6,000 rpm for 1 minute. Subsequently, the resulting solution was poured into 25 ml of a 0.5% PVA aqueous solution and stirred with a homogenizer at 10,000 rpm for 3 minutes, and the resulting solution was poured into 65 ml of a 0.5% PVA aqueous solution and stirred at 700 rpm for 5 hours to give RGD-loaded PLGA nanoparticles. Vancomycin/dexamethasone nanoparticles were prepared in the same manner as Exemplary Embodiment 3. A predetermined amount of each of the RGD-loaded PLGA nanoparticles and the vancomycin/dexamethasone nanoparticles was dispersed in deionized water and then a PGA film was added thereto. The resulting mixture was slowly shaken for 6 hours, and the PGA film was washed three times to remove excess particles from the surface thereof to give a PGA film with a drug delivery system containing three types of bioactive molecules on which the RGD-loaded PLGA nanoparticles and the vancomycin/dexamethasone nanoparticles were immobilized.

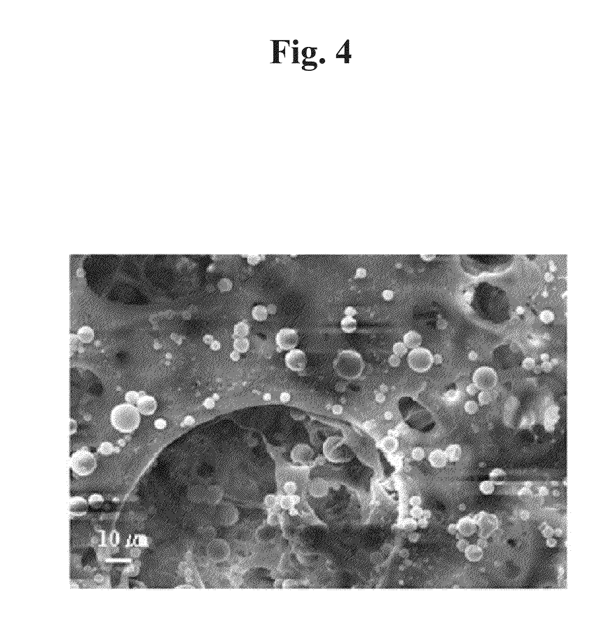

The thus prepared PGA film showed the results similar to those in Exemplary Embodiment 3. As shown in FIGS. 4A and 4B by SEM analysis, it could be confirmed that the RGD-loaded PLGA nanoparticles and the vancomycin/dexamethasone nanoparticles were well dispersed and immobilized on the surface of the PGA film.

### Exemplary Embodiment 5

As a polymeric material for the test, a PLGA scaffold was prepared. Bone morphogenic protein (BMP) and vascular endothelial growth factor (VEGF)-loaded PLGA particles were prepared in the same manner as Exemplary Embodiment 4. Subsequently, the PLGA scaffold was put in distilled water in which PLGA/BMP particles and PLGA/VEGF particles were dispersed in a ratio of 70:30 wt % and the resulting solution was shaken for 6 hours. Then, the PLGA scaffold was washed with distilled water three times to remove excess particles on the surface thereof to give a PLGA scaffold with a drug delivery system containing two-types of bioactive molecules on which the BMP-loaded PLGA and VEGF-loaded PLGA particles were immobilized.

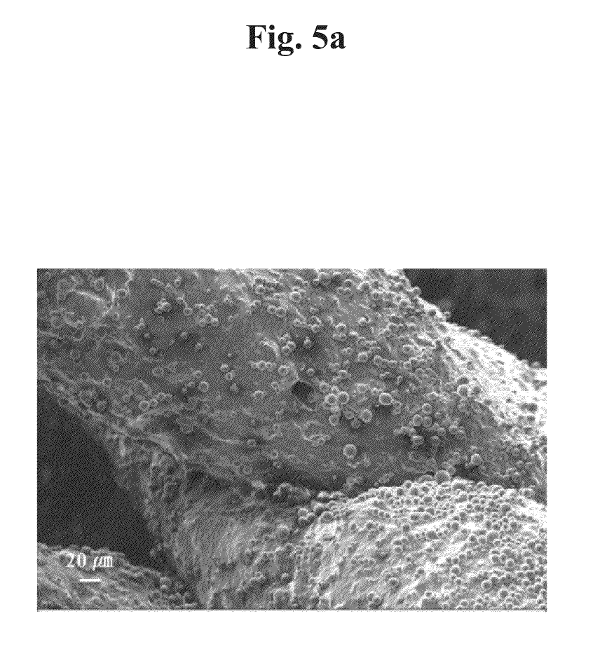

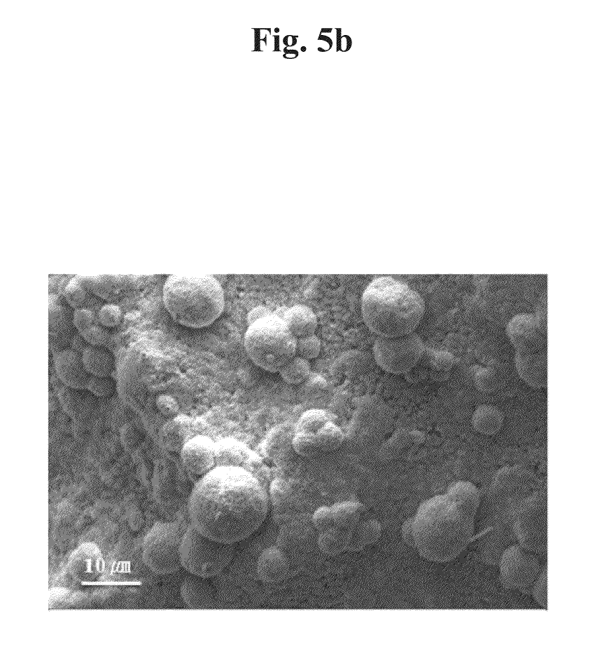

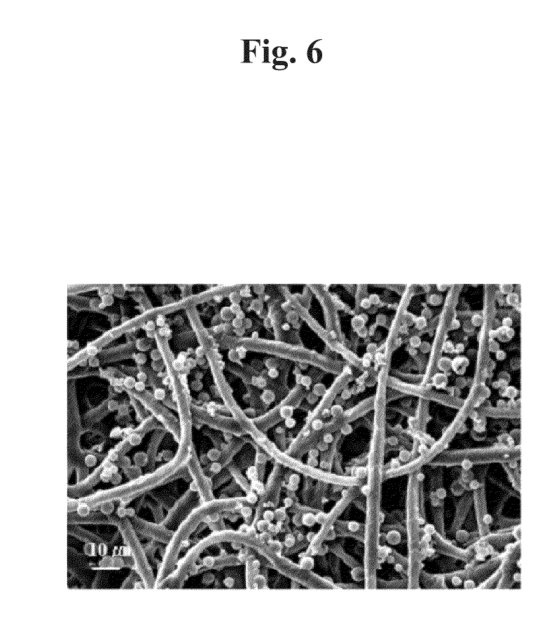

The thus prepared PLGA scaffold showed the results similar to those in Exemplary Embodiments 3 and 4, and it could be confirmed from FIG. 5A by SEM analysis that the BMP-loaded PLGA and VEGF-loaded PLGA particles were well dispersed and immobilized on the surface of the scaffold. As shown in FIG. 5B, it could be also confirmed that the apatite was uniformly formed on the surface of the particle-immobilized scaffold.

### Exemplary Embodiment 6

As a polymeric material for the test, a PCL nanofiber membrane for wound healing was prepared. Nanoparticles by ion complex of BMP/chondroitin sulfate and PEI/dexamethasone were prepared in the same manner as Exemplary Embodiment 3. Subsequently, a predetermined amount of the nanoparticles was dispersed in distilled water and the PCL nanofiber membrane was put therein. The resulting mixture was slowly shaken for 6 hours, and the PCL nanofiber membrane was washed with distilled water three times to remove excess nanoparticles on the surface thereof to give a PCL nanofiber membrane with a drug delivery system containing four-types of bioactive molecules on which the BMP/chondroitin sulfate and PEI/dexamethasone nanoparticles were immobilized.

The prepared PCL nanofiber membrane was analyzed on an SEM, and thus it could be confirmed that the BMP/chondroitin sulfate and PEI/dexamethasone nanoparticles having an average diameter of 900 nm were well dispersed in the pores of the membrane and on the surface of the nanofibers.

### Exemplary Embodiment 7

As a polymeric material for the test, a biodegradable PLA plate was prepared. Fibroblast growth factor (FGF)-loaded PCL nanoparticles and epidermal growth factor (EGF)-loaded PLGA nanoparticles were prepared in the same manner as Exemplary Embodiment 4, and nanoparticles by ion complex of dexamethasone/chondroitin sulfate and BMP/vancomycin were prepared in the same manner as Exemplary Embodiment 3. Subsequently, a predetermined amount of each of the FGF-loaded PCL nanoparticles, the EGF-loaded PLGA nanoparticles, and the dexamethasone/chondroitin sulfate and BMP/vancomycin nanoparticles were dispersed in distilled water, a PLA plate was put therein, and the resulting mixture was slowly shaken for 4 hours. The PLA plate was washed with distilled water three times to remove excess nanoparticles on the surface thereof to give a PLA plate with a drug delivery system containing six-types of bioactive molecules on which the FGF-loaded PCL nanoparticles, the EGF-loaded PLGA nanoparticles, and the dexamethasone/chondroitin sulfate and BMP/vancomycin nanoparticles were immobilized.

The thus prepared PLA plate showed the results similar to those in Exemplary Embodiments 3 and 4, and it could be confirmed that these nanoparticles were well dispersed and immobilized on the surface of the PLA plate.

As described above, the present invention provides a functional polymeric material with a drug delivery system, which can more effectively and easily incorporate a variety of bioactive molecules in to the surface of the polymeric material compared to the conventional methods. The bioactive molecules to inhibit the inflammatory reaction, enhance the wound healing effect, and improve the cytotropism and tissue compatibility can be loaded on particles having a size of several tens of nanometers to several hundreds of micrometers. Since the particles were physically immobilized on the surface of the polymeric material, it can be applied to any implantable biomedical devices regardless of the type of raw material for the polymeric material and the shape and the modified surface of the polymeric material. Moreover, even after the bioactive molecule-loaded particles are immobilized on the surface of the polymeric material, the intrinsic properties of the polymeric material such as mechanical strength, in-vivo breaking strength, degradation rate, porosity, etc. can be maintained. Since the particles can be prepared using any biodegradable polymers or bioactive molecules having a charge, it is possible to effectively control the number, content, and releasing rate of bioactive molecules to be incorporated into the surface of the polymeric material. As a result, it is possible to significantly improve the wound healing effect, cytotropism, and inflammatory reaction at an implantation site by the bioactive molecules, thereby supporting active tissue regeneration.

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A method for preparing a bioactive molecule-loaded implantable polymeric material device with a drug delivery system, the method comprising:
(1) preparing micrometer or nanometer sized bioactive molecule-loaded particles;
(2) modifying the surface of the prepared particles and immobilizing the particles on the surface of the polymeric material; and
(3) physically treating the surface of the polymeric material to improve binding strength of the particles immobilized thereon.

2. The method of claim 1, wherein the implantable polymeric material device comprises at least one selected from the group consisting of silk, cotton, linen, collagen, chitin/chitosan, polydioxanone (PDO), poly(glycolic acid) (PGA), polylactic acid (PLA), poly(ε-caprolactone) (PCL), lactide-co-glycolic acid (PLGA), glycolide-co-trimethylene carbonate (GA-TMC), glycolide-co-ε-caprolactone (GA-CL), polyglyconate (PGC), polyglactin (PG), polyamino acid, polyanhydride, polyorthoester, a polyester group, a polyether group, a polyethylene group, a polypropylene group, a polybutester group, a polytetrafluoroethylene group, a polyamide group, a polyimide group, polyvinylidene fluoride, and copolymers thereof, stainless steel, titan, nitinol, silver, and gold.

3. The method of claim 1, wherein the implantable polymeric material device is in the form of a block, a film, a filament, a fiber, non-woven/woven, a membrane, a mesh, a knit, a granule, a particle, a plate, a bolt/screw, a pin, or a complex thereof.

4. The method of claim 1, wherein the implantable polymeric material device is applied to suture and ligature, guided bone regeneration (GBR) and guided tissue regeneration (GTR), and human soft and hard tissue regeneration of skin, gum and gum bone, blood vessel, bone, muscle, tendon and artificial organs.

5. The method of claim 1, wherein in step (1), the particles comprise at least one selected from the group consisting of polydioxanone (PDO), poly(glycolic acid) (PGA), polylactic acid (PLA), poly(ε-caprolactone) (PCL), lactide-co-glycolic acid (PLGA), glycolide-co-trimethylene carbonate (GA-TMC), glycolide-co-ε-caprolactone (GA-CL), polyglyconate (PGC), polyglactin (PG), polyamino acid, polyanhydride, polyorthoester and copolymers thereof; collagen, gelatin, chitin/chitosan, alginate, albumin, hyaluronic acid, heparin, fibrinogen, cellulose, dextran, pectin, polylysine, polyethyleneimine, dexamethasone, chondroitin sulfate, lysozyme, DNA, RNA, protein derivatives and copolymers thereof; a growth factor, a growth hormone, a peptide drug, a protein drug, an anti-inflammatory and analgesics drug, an anti-cancer drug, an anti-viral drug, a sex hormone, antibiotics, antimicrobials and compounds thereof; and metals such as gold, silver and zinc.

6. The method of claim 1, wherein in step (1), the particles are non-porous or have a porosity of 5 to 98% and a diameter of 10 nm to 1 mm.

7. The method of claim 1, wherein in step (1), the particles are prepared by a water/oil or water/oil/water emulsion method, a spray method, a phase separation method, or a polyelectrolyte complex method.

8. The method of claim 1, wherein in step (1), the bioactive molecules are selected from the group consisting of a growth factor, a growth hormone, a peptide drug, a protein drug, an anti-inflammatory and analgesics drug, an anti-cancer drug, an anti-viral drug, a sex hormone, antibiotics, antimicrobials, and a compound thereof.

9. The method of claim 8, wherein the bioactive molecules are selected from the group consisting of growth factors such as a transforming growth factor, (TGF), a fibroblast growth factor (FGF), a bone morphogenic protein (BMP), a vascular endothelial growth factor (VEGF), an epidermal growth factor (EGF), an insulin-like growth factor (IGF), a platelet-derived growth factor (PDGF), a nerve growth factor (NGF), a hepatocyte growth factor (HGF), a placental growth factor (PIGF), and a granulocyte colony stimulating factor (G-CSF); peptide and protein drugs such as heparin, porcine growth hormone (pGH), human growth hormone (hGH), erythropoietin, (EPO), a granulocyte colony stimulating factor (gCSF), interferon (INF), follicle stimulating hormone (FSH), luteinizing hormone (LH), goserelin acetate, leuprorelin acetate, triptorelin acetate, and luteinizing hormone-releasing hormone agonist (LH-RH agonist); anti-inflammatory and analgesics drugs such as dexamethasone, indomethacin, ibuprofen, ketoprofen, piroxicam, flurbiprofen, and diclofenac; anti-cancer drugs such as paclitaxel, doxorubicin, camptothecin, 5-fluorouracin, cytosine arabinose, and methotrexate; anti-viral drugs such as acyclovir, Robavin, and Tamiflu; sex hormones such as testosterone, estrogen, progesterone, and estradiol; antibiotics such as tetracycline, minocycline, doxycycline, ofloxacin, levofloxacin, ciprofloxacin, clarthromycin, erythromycin, cefaclor, cefotaxim, imipenem, enicillin, gentamicin, streptomycin, and vancomycin; anti-fungal drugs such as ketoconazole, itraconazole, fluconazole, amphotericin-B, mystatin, and griseofulvin; and compounds such as β-glycerophosphate, ascorbate, hydrocortisone, and 5-azacytidine.

10. The method of claim 1, wherein in step (1), the bioactive molecules are used in the range from 10⁻⁷ to 100% with respect to of the total weight of the particles.

11. The method of claim 1, further comprising modifying the surface charge of the particles or the polymeric material device prepared in step (2) to be opposite to each other and allowing the particles to be physically immobilized on the surface of the surface of a suture by electrostatic interaction.

12. The method of claim 11, wherein the modifying of the surface charge comprises coating the surface of the particles or the polymer material device with a material having a charge.

13. The method of claim 12, wherein the material having a charge comprises at least one selected from the group consisting of polydioxanone (PDO), poly(glycolic acid) (PGA), polylactic acid (PLA), poly(ε-caprolactone) (PCL), lactide-co-glycolic acid (PLGA), glycolide-co-trimethylene carbonate (GA-TMC), glycolide-co-ε-caprolactone (GA-CL), polyglyconate (PGC), polyglactin (PG) and copolymers thereof, collagen, heparin, albumin, hyaluronic acid, dextran, vancomycin, chitosan, dexamethasone, chondroitin sulfate, lysozyme, polylysine, polyethyleneimine (PEI), sodium tripolyphosphate (TPP), polystyrene sulfonate (PSS), polyallylamine (PAAm), polyvinylamine (PVAm), poly(diallyldimethylammonium chloride) (PDADMAC), poly(methylamino) ethyl methacrylate (PDAMAEMA), N-hydroxysuccinimide (NHS), N-3-dimethylaminopropyl-N′-ethyl-carbodiimide hydrochloride (EDC), and copolymers thereof.

14. The method of claim 1, wherein step (2) comprises partially dissolving the particles or the surface of the polymeric material device using a solvent to immobilize the particles on the surface of the polymeric material.

15. The method of claim 14, wherein the solvent comprises at least one selected from the group consisting of water, hydrochloric acid, acetic acid, methylene chloride, ethanol, acetone, methanol, dichloromethane, chloroform, toluene, acetonitrile, 1,4-dioxane, tetrahydrofuran, hexafluoroisopropanol or a mixed solvent thereof.

16. The method of claim 1, wherein step (2) comprises inserting the particles into pores and gaps of the polymeric material device without any surface modification of the particle.

17. The method of claim 16, wherein the particles inserted into the pores and the gaps of the polymeric material device are immobilized by post treatment.

18. The method of claim 17, wherein:
step (2) comprises partially dissolving the particles or the surface of the polymeric material device using a solvent to immobilize the particles on the surface of the polymeric material;
the solvent comprises at least one selected from the group consisting of water, hydrochloric acid, acetic acid, methylene chloride, ethanol, acetone, methanol, dichloromethane, chloroform, toluene, acetonitrile, 1,4-dioxane, tetrahydrofuran, hexafluoroisopropanol or a mixed solvent thereof; and
in the post treatment, the solvents mixed in 0 to 100 wt % are used.

19. The method of claim 17, wherein in the post treatment, the particles are immobilized by heat of 30 to 300° C.

20. The method of claim 1, wherein in step (2), the particles or the surface of the polymer material device is modified with plasma using a gas such as argon, oxygen, hydrogen peroxide or ammonia to improve hydrophilicity and ionic activation thereof.

21. The method of claim 1, wherein step (3) comprises using a solvent or heat capable of dissolving the particles or the surface of the polymeric material device to improve binding strength of the particles immobilized on the surface of the polymeric material device.

22. The method of claim 21, wherein:
the solvent is mixed in 0 to 100 wt %; and
the particles are immobilized by heat of 30 to 300° C.

23. The method of claim 1, wherein step (3) comprises immersing the polymeric material device in a simulated body fluid (SBF) solution within 2 days by an alternate dipping process to form apatite on the surface of the polymeric material device.

24. The method of claim 1, wherein in step (3), the SBF solution has an ion concentration of 1 to 5 times.

25. An implantable polymeric material device with a drug delivery system using the bioactive molecule-loaded biodegradable polymeric particles and the particles composed of bioactive molecules obtained by the method of claim 1.
